(19) United States
(12) Patent Application Publication
(10) Pub. No.: US 2021/0151139 A1
(43) Pub. Date: May 20, 2021

(54) Enhanced Patient Registration Process software

(71) Applicant: Melissa Bloom, Clinton, MS (US)

(72) Inventor: Melissa Bloom, Clinton, MS (US)

(21) Appl. No.: 16/684,485

(22) Filed: Nov. 14, 2019

(51) Int. Cl. G16H 10/60; G16H 10/20; G06Q 40/08; G06Q 40/02; G06Q 10/10; G06K 9/20

(52) U.S. Cl. CPC G16H 10/60; G16H 10/20; G06Q 40/08; G06F 3/0482; G06Q 10/10; G06K 9/2054; G06Q 40/02

(57) ABSTRACT

Enhanced Patient Registration Process software integrates a scanner, tablet and exiting practice management system. The software takes the input received from the tablet and reads the scanned cards to locate information needed to create a complete and whole patient demographic and insurance profile by mapping the data received from the tablet and scanned cards directly into the practice management system with less than 10% human interaction. The software guides patient's through the patient check in/registration process and requests data from the patient via the tablet searching for specific information consideration all things need by the front desk representative and the medical biller. The software is embedded with the knowledge of both a front desk representative, medical biller and the unique insurance company's requirements ensuring that at the end of the guided process the patient profile is complete and ready to receive billable charges.

### CROSS-REFERENCE TO RELATED APPLICATIONS

N/A

### STATEMENT REGARDING FEDERALLY SPONSORED RESEARCH OR DEVELOPMENT

N/A

### BACKGROUND OF THE INVENTION

The intent of this invention, Enhanced Patient Registration Process software is to use this uniquely written software, Enhanced Patient Registration Process software, to minimize, enhance and improve the accuracy of the time consuming “check in” process that front desk personnel in all medical facilities endure. There is an exchange of paperwork between patient and personnel, usually 4-5 pages. Once the paperwork is completed by the patient and then reviewed for errors by the front desk person, it's then entered manually by the personnel. The extent of collecting all the needed data, checking for errors and entering the data properly is highly contingent on the knowledge level of personnel. This process is vital because accurate and complete patient demographic and insurance information is required in order for healthcare professionals to receive reimbursement for the services performed. The Enhanced Patient Registration software improves upon prior art by scanning, extracting, thinking, manipulating and organizing the data where it belongs directly into the practice management system on the computer like a front desk worker and a medical biller with extensive knowledge of billing and patient registration specific to a medical office would . This software goes beyond making and storing a copy. This software goes beyond extracting the data and placing it in a database outside of the practice management software from reentry or copy and pasting at a later time. It improves upon prior art by making the patient and/or front desk person aware of what's lacking in the patient's profile based upon the data is has collected and aggregated. The Enhanced Patient Registration Process software will have the ability to allow communication between the practice management system on the computer, the scanner and a tablet. The software integration will also include a component that allows for the same unique scan, extract and think capability for insurance cards and identification cards that are emailed. This is to accommodate the rise in electronic insurance cards only made available via computer or mobile application.

When insurance cards are only made available electronically, it comes with the option to email a clean digital copy out for registration purposes. The software will also include language for a tablet that allows patients to enter their own data updates that cannot be extracted from another source via scanner. There are patient kiosks on the market now that will allow the patient to scan their medical and identification cards. With these kiosks, once the data is scanned and stored, it is assigned or sent to another person to properly place the data manually into its final destination which is the practice management system. Likewise some prior art also allows the patient to enter what is deemed the most important information, the insurance details, themselves in to a mobile device. The Enhanced Patient Registration Process software will greatly reduce, or practically eliminate the need of medical office staff interaction and greatly improve the accuracy of pertinent data entered directly into the Practice Management system. The Enhanced Patient Registration Process software will absorb those duties. The Enhanced Patient Registration Process software will be interfaced to existing practice management systems already in use on the market like Practice Fusion, Office Ally and NueMD to name only a few. This is important because prior art which does provide any improvement to the vital process of patient registration and check in limits the medical offices and practice management systems that have access to it. The software will work with most input devices, scanner and tablets, already utilized by the office as a consideration allow more and more medical offices to benefit from Enhanced Patient Registration Process software.

### BRIEF SUMMARY OF INVENTION

When patients enter a clinic or hospital, they are required to check in at a front desk before being seen by the health provider. The process is currently 90% manual for most medical facilities. This check in and registration process is necessary because accurate and complete patient demographic and insurance information is required in order for healthcare providers to receive reimbursement for the services performed. Enhanced Patient Registration Process software improve, enhance, streamline and simplify this process by enabling communication between the practice management system on the computer, the data entry tablet device and the scanner that will allow a continuous flow of extracting and requesting information with the guidance of the software until a complete, whole and accurate patient profile is created in the interfaced practice management system. Enhanced Patient Registration Process software uses tablet to communicate data requests to the patient and the scanner to receive, extract and organize data all the vital, sensitive information needed directly from the identification and insurance cards for a complete patient demographic and insurance profile. All entered and scanned data will be communicated directly into the practice management system. The software will load on any major brand scanner and tablet currently used by the facility/office, and the practice management software. The programming language will vary because there are many practice management systems in use now for healthcare providers. The software integration will also include a component that allows for the same unique scan and think capability for insurance cards and identification cards that are emailed. The software eliminates the need for 90% medical office staff understanding and data entry and removes common errors made when performing this task manually. The extraction of data from the insurance and identification cards is more in depth than just insurance company name, policy number, policyholder name and other surface details. It looks for details needed not only for front desk but details needed by the medical biller as well. The software will be able to scan and “think” in a way that a front desk and a medical biller with in depth knowledge would. This means reading the card more so than simply scanning and copying what is there. The software will read the card and place the information where it belongs in the actual practice management system on the computer during the extraction process.

The Enhanced Patient Registration Process software is critical and innovative as it will know what key words , data formats and data placement to look for, as well as what additional information to request if not available on the card. Additionally, the software will know what additional details are needed from the patient based on the patient's insurance profile and what each individual insurance company requires. It will also map this data to the appropriate field or make the appropriate selection from the drop down menu directly in the practice management system on the computer versus in a database outside of the system that would require additional movement and manipulation by an individual. The tablet will allow the patient to make manual updates using a set of guided commands given by the Enhanced Patient Registration Process software based on the condition of patient's profile as well as the selections made by the patient. For example, an existing patient may need to make an address update and the driver's license or any other source does not have it to scan from. The tablet will allow direct entry or it can be updated manually by the office personnel. Any data entered by the patient on the tablet will be mapped directly into the practice management system on the computer as well, Some of the immediate advantages are decreased time spent manually entering data, decreased check in time, optimal efficiency and increased accuracy. Most Individual and privately held practices are limited in resources and staff. The knowledge and understanding needed to properly obtain and store pertinent data for proper medical billing, which is the ultimate goal, is rarely found in one person. Typically the jobs are divided, but they are very much intertwined. This software will allow the task to be done correctly, efficiently and accurately with little to less than 10% medical office staff intervention or knowledge required. An additional benefit that can also control cost is that the software will work with any major brand scanner and tablet and does not require a specific duplex printer like prior art developed.

### BRIEF DESCRIPTION OF DRAWINGS

FIG. 100: Description of the software AI workflow.

### DETAILED DESCRIPTION OF THE INVENTION

Enhanced Patient Registration Process software will scan, read, request and map pertinent data directly into various existing practice management systems like Practice Fusion, Office Ally and NueMD. The information can be scanned directly from the identification and insurance card or emailed copies of the same cards. Any major brand of scanner can be used, single scan or duplex. The software will also use a major brand tablet (mobile device) to allow patients to make manual updates and enter information that cannot be scanned, but is necessary for a complete and accurate patient demographic and insurance profile , as prompted by the software interfaced with the practice management system. The software thinks using the embedded programming language like a front desk person and a medical biller in what it looks for on the scanned cards. The software also knows what to request from the patient if it is not available on the scanned identification and insurance cards, based on the data is has collected and aggregated. The software organizes data directly into the practice management system on the computer, enabling communication between the input devices and the practice management software. What is unique about this software is that it is written with enhanced mapping logic programming language to think versus just scan and copy. It looks for and extracts data in a unique way. It also looks beyond the surface data which is patient demographics, insurance payer name and policy number and looks for medical billing and insurance details. It will work with a tablet as an input interface to collect data directly from the patient based on the data scanned or entered. It will recognize when pertinent data is missing or outdated in the practice management system by way of staff notation or empty fields within the patient profile. The software will make requests on the tablet for the patient based on the appropriate algorithm. For example, if mailed correspondence was returned and the mailing address fields are empty, the software will recognize that and request an update from the patient. As second example is if the insurance policy holder is someone other than the patient, the software will know to request additional information about the policyholder from the patient because the software knows that this is a requirement of the insurance company. The software will greatly decreases human interaction for this task which saves time and money. It also increases accuracy and efficiency by way completely removing human error.

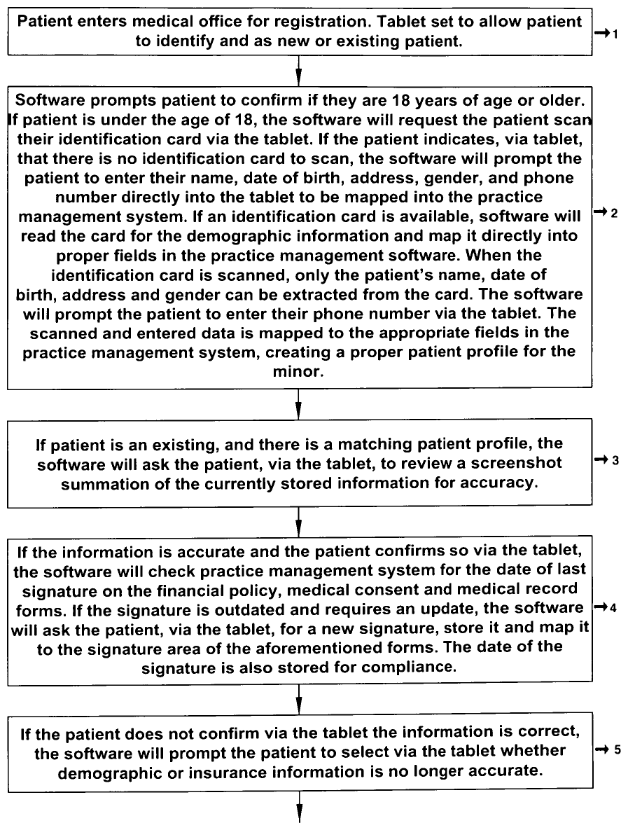

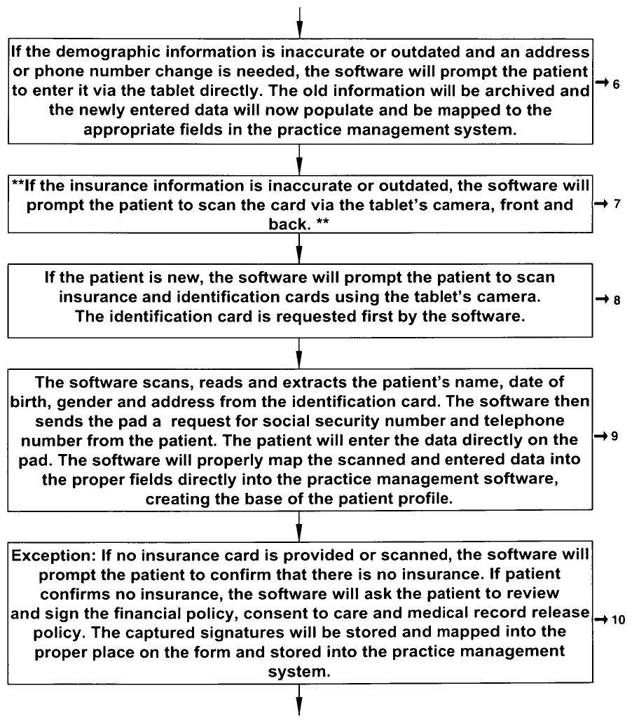

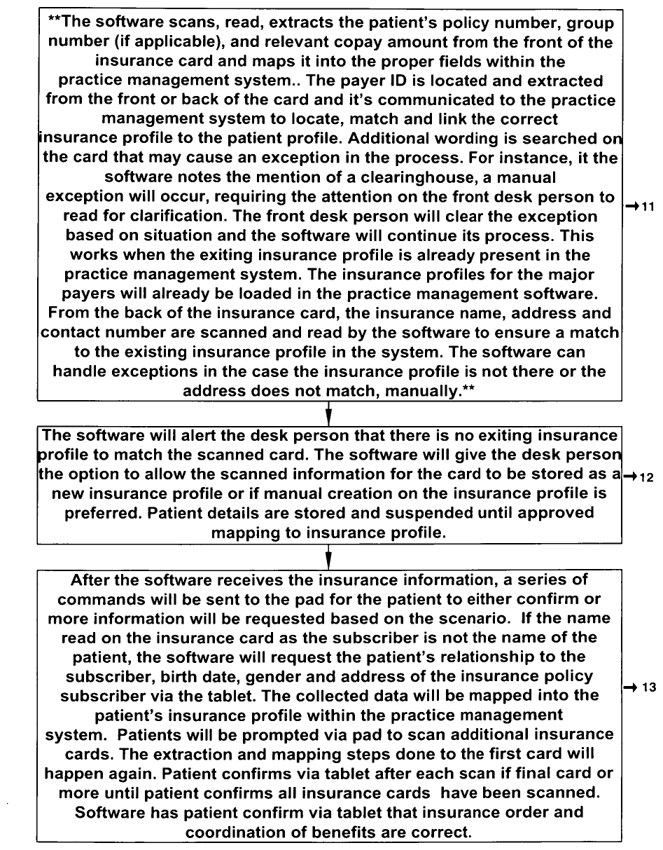

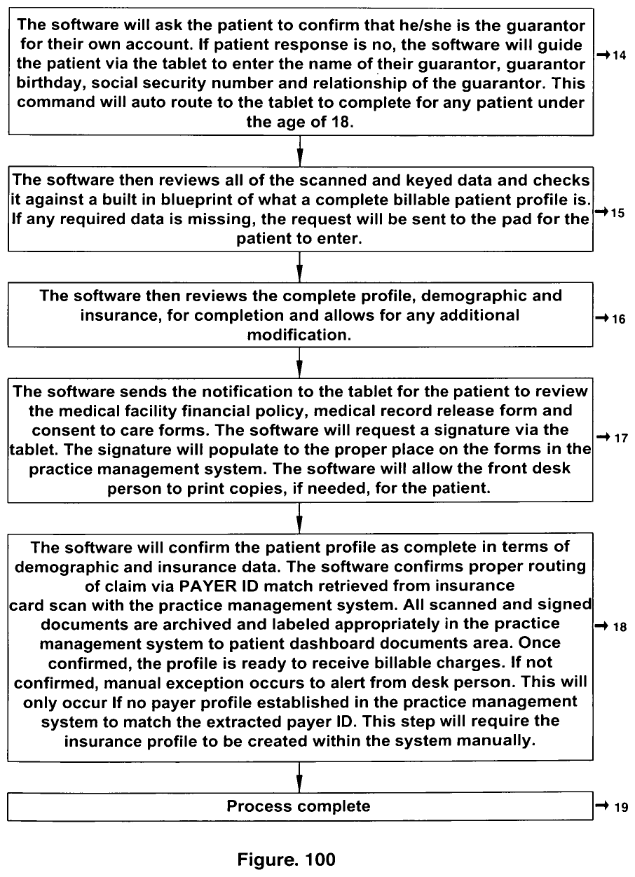

FIG. 100: shows Description of the software AI workflow and step 1-19 shows the execution of process and steps involved.

The way Enhanced Patient Registration Process works is detailed below:

1. Patient enters clinic and goes to front desk to be newly registered or revalidated (new patient or existing patient) prior to being seen.

2. For new patients, the patient profile will be established by the initial scan of the identification card. The medical office's resources will determine if this is done by way of the patient handing the cards to the front desk person to scan or if the medical office will allow patient's to scan their own cards using the tablet's camera. The software Is written to work with multiple major brand scanners. The scanner does not have to be a duplex scanner as required in some prior technology. If the scanner is not duplex, it will require the front desk person or patient to simply flip the insurance card being scanned. For insurance cards especially, scanning the front and back of the card is pertinent.

3. The government issued identification card should be scanned first. The software will extract the name, date of birth, address and gender. The software will scan and read the data. It will the map and organize the information from the identification card to the field that corresponds within the practice management system. The coding language uses language that will make selections in the practice management software based on certain criteria and wording, data format and data position on the card. When the insurance card is scanned, it will extract all the billing and insurance company details. It is important to note here that the coding language will include the many variances, formats, layouts and text identifiers used on insurance cards. Insurance cards are formatted differently and reference data sets by different names. An example is the policy number may be placed behind the word POLICY one card and then placed behind ID # on the other. The software will use its read capability and language to identity the proper data regardless of card type. For adolescent patients with no identification, all of the demographic information will have to be entered after the prompt.

4. Government issued identification card to Practice Management Software Mapping
Position1/xxxxxx, xxxxxx sequenced mapped to Last Name, then First Name
2/DOB xx/xx/xxxx mapped to DOB field-can be in-versed is system requires it
3/numeric/text
xxxxxxx, xx, xxxxx sequence mapped to Address, State and Zip Code
Sex returns Female/F or Male/M mapped to Gender selection M or F

5. Additional pertinent data that may be required based on medical office's policy are the social security number and a telephone number. The software will prompt the patient to enter the data via the tablet.

6. Insurance Card to Practice Management Software Mapping (BOTH SIDES OF THE CARD WILL BE SCANNED)
Policy/ID mapped to Policy Number field
Grp/group mapped to Group Number field
Copay $xx mapped to Copay field-search for words office, specialty
Payer xxxxx Payer ID xxxxx mapped to back end billing to connect to the insurance profile
payer service number mapped to individual insurance contact profile
payer address and phone number scanned to ensure match to existing insurance profile in practice management system.

7. As with the identification card, the scanned insurance card, based on what the software reads, may prompt the request of additional information needed. If the software reads that the subscriber name does not match the patient name, the software will prompt the pad to request the patient's relationship to the subscriber, the subscriber's date of birth, gender and address. The tablet will ask if additional, secondary insurance card is relevant. If yes, step 5 will repeat.

8. Using the tablet, the software will ask the patient to confirm that he or she is the guarantor for the account. If the patient does not confirm, the software will request the name, relationship, date of birth, social' security number of the account guarantor. For any patients under the age of 18, the software will use the tablet to prompt the patient to enter this data automatically regardless of prior selections.

9. The software will then ask the patient to review financial policy, medical record release policy and consent to care forms. The patient's signature will be requested on the tablet and populated in the mapped signature line of each form. The front desk person will be given the option to print any signature forms at patient's request.

10. All scanned and keyed data will be aggregated and checked for completeness between software language and patient profile. The result will be a complete patient profile ready to receive billable charges. The patient will be given confirmation of a complete profile.

11. Existing patients will have the option via tablet and a precisely directed set of commands made by the software to confirm last data on file is accurate OR to indicate if an update is necessary. If no update is necessary , the software will check date of last signature on file. If in excess of 365 days. The tablet will prompt the patient to review the financial policy, medical record release policy and consent to care policies and then request a signature on the pad that will map to the correct areas of the signature page in the practice management system.

12. If an update is needed, the software will prompt the patient via tablet to scan the insurance card or the card can be provided to the front desk person to scan. The software will read the scan and extract and map all the data in the practice management system on the computer. The same series of events will happen as outlined in the new patient process, steps 4-6.

13. If the update is to the address, the software will prompt the patient to enter the new address on the tablet. The alternative is that the address is actually correct on the identification card which means it can be scanned. The software will queue the option for self-scan or medical office staff to scan. Steps 3-4, 9 will be repeated.

14. The Software includes a blueprint of what a complete billable patient profile looks like for all insurance companies.

15. All scanned images will be saved in the practice management system in the appropriate place for later reference is needed.

1. The Enhanced Registration Process software invention's claim is to perform the complete patient registration process inclusive of demographics and insurance profile data requiring less than 10% medical office staff intervention and task knowledge-essentially absorbing the manual duties of a very knowledgable front desk person and medical biller using steps comprising of:
a. Programming language that is embedded with the logic of tasks performed by a front desk and medical billing person
b. Enabling communication between existing practice management systems, a scanner and a tablet;
c. Aggregating the data received via scanner and tablet to determine if additional data is needed that can be not be simply scanned and requesting that data directly from the patient via tablet;
d. Scanning, reading and extracting all pertinent data from the insurance card ensuring the capturing of the payer ID, insurance policy number and relative copay to map directly into the practice management system, identify and link to the correct insurance company profile from within the practice management system, the copay and to verify the insurance company's address and telephone number match the exiting payer profile within the practice management system;
e. Mapping and organizing all extracted, keyed and entered data directly into the corresponding fields in the practice management system in its required format;
f. Aggregating and analyzing all extracted and received data is a based on software logic and language to detect any missing data pertinent to complete the patient profile, demographic and insurance;
g. Recognizing any missing data or additional data required based on scanned or patient entered information and/or unique specification of the insurance company and requesting it via integrated tablet for patient to provide before finalizing the patient profile as complete;
h. Ensuring proper claim submission to correct insurance company/payer;
i. Ensuring a whole, completely accurate patient demographic and insurance profile ready for medical, upon completion of software process;

2. The Enhanced Patient Registration Process software of claim 1 further claims is to scan, compare, read, extract, organize and prompt data and subsequently map all extracted and requested data into the practice management system needed to create a whole and complete patient profile ready to receive billable charge to submit to any insurance company or payer via total automation;

3. The Enhanced Patient Registration Process software of claim 1 further claims to be able to accommodate and integrate with multiple practice management systems on the market;
